(19) United States
(12) Patent Application Publication
(10) Pub. No.: US 2016/0084842 A1
(43) Pub. Date: Mar. 24, 2016

(54) WOVEN HYDROGEL BASED BIOSENSOR

(71) Applicant: Syracuse University, Syracuse, NY (US)

(72) Inventors: Yan-Yeung Luk, Jamesville, NY (US); Mark Weldon, Liverpool, NY (US); Gauri Shetye, Syracuse, NY (US); Andrew Basner, Syracuse, NY (US); Karen Simon, Cambridge, MA (US); Erik Burton, Phoenix, NY (US)

(73) Assignee: Syracuse University, Syracuse, NY (US)

(21) Appl. No.: 14/953,606

(22) Filed: Nov. 30, 2015

Related U.S. Application Data
(62) Division of application No. 13/754,935, filed on Jan. 31, 2013.
(60) Provisional application No. 61/592,942, filed on Jan. 31, 2012.

(51) Int. Cl. G01N 33/58

(52) U.S. Cl. CPC G01N 33/582

(57) ABSTRACT

A porous hydrogel sensor that is responsive to the presence of one or more target compounds in solution is synthesized based on demixing of certain molecules in the presence of a target compound. The porous hydrogel sensor may include fluorescently tagged antibodies that are noncovalently bound to the gel and then released in the presence of the target antigen. The porous hydrogel sensor may alternatively include dissolvable cross-links using polymerized antibody and antigen complexes so that, in the presence of the target antigen, the cross-links will be displaced and the hydrogel will dissolve.

### CROSS-REFERENCE TO RELATED APPLICATIONS

The present application is a divisional application of U.S. application Ser. No. 13/754,935, filed on Jan. 31, 2013, which claimed priority to U.S. Provisional Application No. 61/592,942, filed on Jan. 31, 2012, both of which are hereby incorporated by reference.

### STATEMENT REGARDING FEDERALLY SPONSORED RESEARCH OR DEVELOPMENT

This invention was made with government support under contract no. 0727491 awarded by the National Science Foundation (NSF) and contract number X-83232501-0 awarded by the Environmental Protection Agency (EPA). The government has certain rights in the invention.

### BACKGROUND OF THE INVENTION

1. Field of the Invention

The present invention relates to biosensors and, more particularly, to a woven hydrogel capable of detecting multiple chemical targets.

2. Description of the Related Art

The rapid and accurate detection of target compounds is needed in a variety of fields. For example, in the medical field, such detection is required for diagnosing the type of disease. With respect to anti-terrorism, the identifying of target compounds is needed to detect and avoid potential toxins, such as chemical and biological weapons. Finally, in the water industry, the rapid and accurate detection of water-related problems, such as the presence of infectious diseases, is required to maintain and protect the available of potable water. Unfortunately, conventional sensor technologies are surface-based, and require elaborate instrumentation and relative long detection times. Moreover, these sensors only provide a “yes” or “no” signal rather than reporting the precise amount of the targeted compound.

### BRIEF SUMMARY OF THE INVENTION

The present invention comprises a porous hydrogel sensor that is responsive to the presence of one or more target compounds in solution based demixing of certain molecules in the presence of a target compound. In a first embodiment, the porous hydrogel sensor includes fluorescently tagged antibodies and antigens that are noncovalently bound to the gel. The fluorescently tagged antibodies are released from the gel when the target antigen is present in solution, thereby providing a visual indication of the presence of the target. In a second embodiment, the porous hydrogel complex is cross-linked using polymerized antibody and antigen complexes. In the presence of the target antigen, the cross-links fail, thus causing the hydrogel to dissolve and providing a simple visual indication that the target compound is present.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a three dimensional schematic of a hydrogel based sensor for detecting a single target according to the present invention;

FIG. 2 is a schematic showing the fabrication of a hydrogel based sensor for detecting a single target according to the present invention

FIG. 3 is a two dimensional schematic of a hydrogel based sensor according to the present invention;

FIG. 4 is a graph illustrating the selective detection of a target by a hydrogel based sensor according to the present invention;

FIG. 5 is a graph illustrating the efficacy of a hydrogel based sensor according to the present invention after refrigeration overnight;

FIG. 6 is a three dimensional schematic of a hydrogel based sensor for detecting multiple targets according to the present invention;

FIG. 7 is a two dimensional schematic of a hydrogel based sensor for detecting multiple targets according to the present invention;

FIG. 8 is a schematic of the process for modifying and polymerizing an antigen/antibody complex for use in a hydrogel based sensor according to the present invention;

FIG. 9 is a schematic of the process for fabricating an antigen-antibody woven hydrogel according to the present invention;

FIG. 10 is a series of chemical diagrams of certain components of a hydrogel based sensor according to the present invention;

FIG. 11 is a schematic of an alternate embodiment of a hydrogel based sensor according to the present invention;

FIG. 12 is a schematic of an alternate embodiment of a hydrogel based sensor having a label free mechanism for detection of targets; and

FIG. 13 is a graph illustrating the selective detection of a target by a hydrogel based sensor according to the present invention.

### DETAILED DESCRIPTION OF THE INVENTION

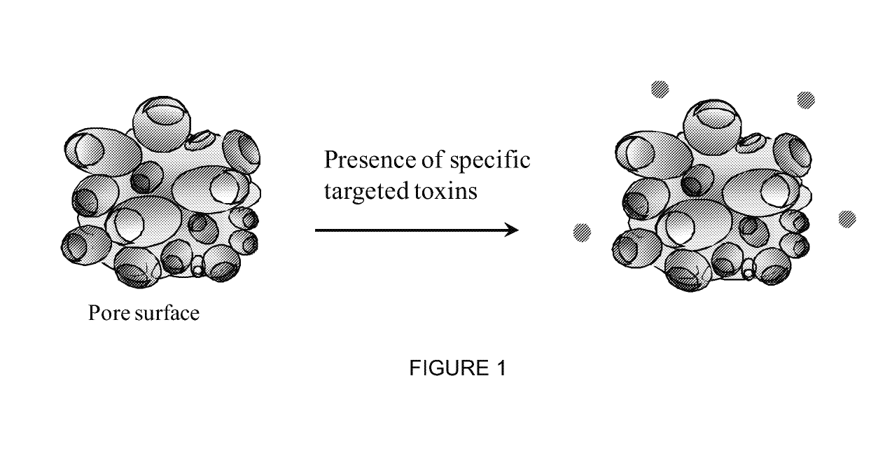

Referring now to the drawings, wherein like reference numerals refer to like parts throughout, there is seen in FIG. 1 a hydrogel based biosensor according to the present invention. The present invention is based on the discovery that certain molecules will demix or separate from each other in water, even though individually the molecules are entirely soluble in water. This separation enables a fabrication of unprecedented new gel materials that can immobilize protein only on the gel surfaces. The immobilized proteins are demonstrated to be highly active in binding to their targeted ligands. The bound ligands can be displaced if there are further ligands in the solution. The displacement reaction of the present invention is highly selective for only the targeted ligand or molecules, and may be implemented as a sensor in at least two different ways.

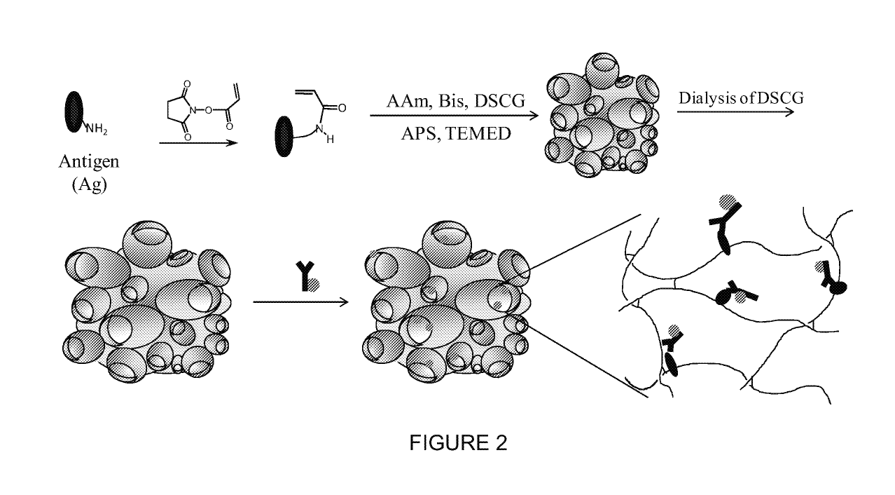

In a first embodiment, a fluorescently tagged antibody is released from a porous gel material where the functional components (antibodies and antigens) are located at desired locations in the gel and are noncovalently bound to the gel. As seen in FIG. 2, the hydrogel is formed by modifying the appropriate antigen with a polymerizable acrylamide group as a monomer for making linear polyacrylamides with antigen side chains. This modification of antigen was done by coupling the lysine groups of the antigen with N-succimidylacrylate (NSA) in phosphate buffer saline (PBS, 10 mM, pH 7.4) at 25° C. for 1 h. The acryloyl-modified antigen was copolymerized with acrylamide (AAm) monomer to generate a covalently crosslinked antigen laden porous hydrogel by mixing initiator APS, catalyst TEMED, crosslinker bisacrylamide and disodium cromoglycate (DSCG) at 25° C. for 12 h. The DSCG was removed through diffusion by soaking the hydrogel in PBS buffer. This dialysis generated an antigen laden swollen porous hydrogel. Fluorescently tagged antibody was immobilized by soaking the antigen laden hydrogel in a solution of fluorescently tagged antibody.

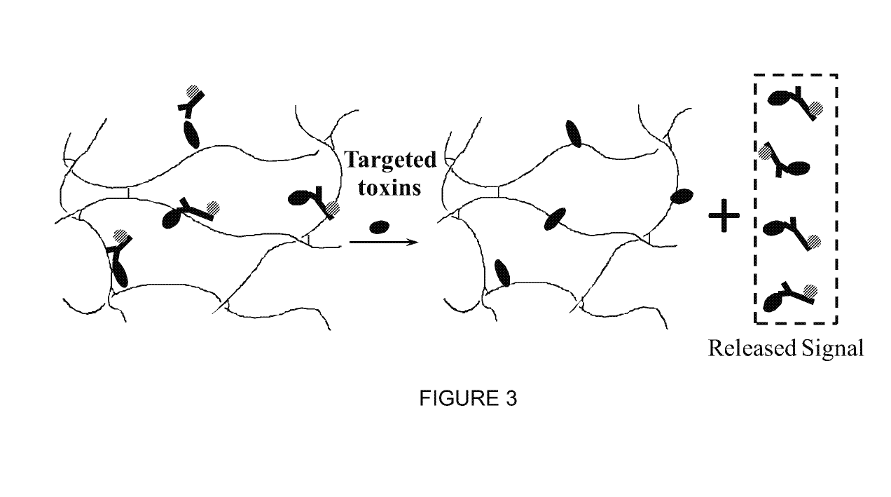

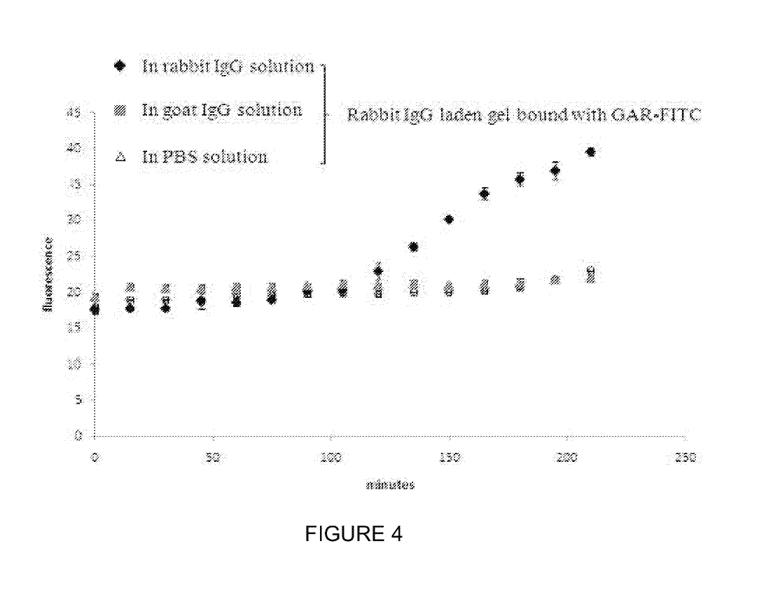

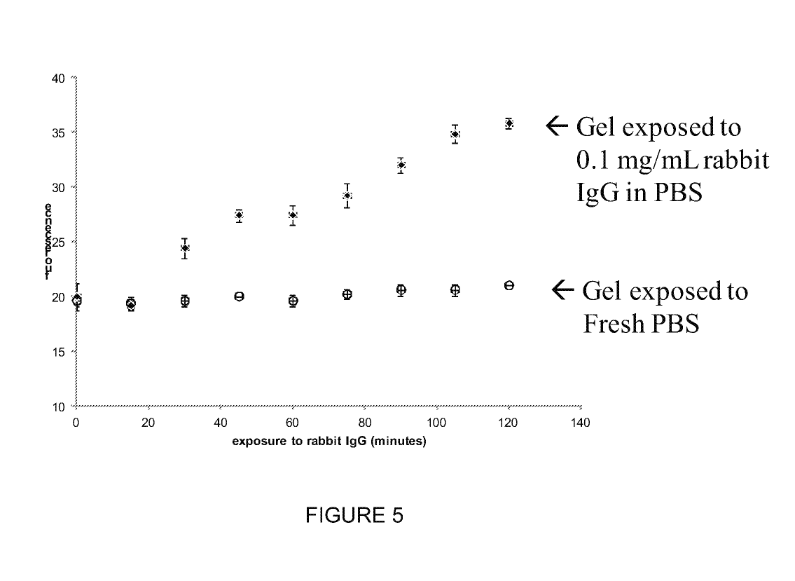

As seen in FIG. 3, in the presence of the targeted antigen in solution will result in the displacement of the non-covalently bound antigen, thereby releasing the fluorescently tagged antibody. This embodiment provides a rigorous quantification method for concentration of the targeted analyte that is an improvement over existing methods. For example, as seen in FIG. 4, a sensor according to this embodiment of the present invention is highly selective (no false positives) for targeted toxins where the target comprises rabbit IgG and the potential false positive compound is goat IgG. As evident from the increase in fluorescence, the rabbit IgG in solution binds and displaces GAR-FITC from gel, while the goat IgG has no binding activity with GAR-FITC. Referring to FIG. 5, an exemplary gel sensor according to the present invention will also retain its high selectivity after storage.

As seen in Table 1 below, a hydrogel sensor according to the present invention represents an improvement over existing detection methods, such as the Enzyme-Linked Immunosorbent Assay (ELISA). The detection time of a sensor according to the present invention about 4-8 times faster (30 minutes versus 3-4 h) than the current methods while requiring only a single step.

TABLE 1

| | Gel Sensor | ELISA |
|---|---|---|
| Quantifiable | In Solution, rigorous | On Surface, Not rigorous |
| Detection time | 30-40 minutes | 2-4 hours |
| Selectivity | High | High |
| Procedures | Single-step | Multiple steps |
| Instrumentation | Can be portable | Can be portable |
| Label-free | Not yet | No |

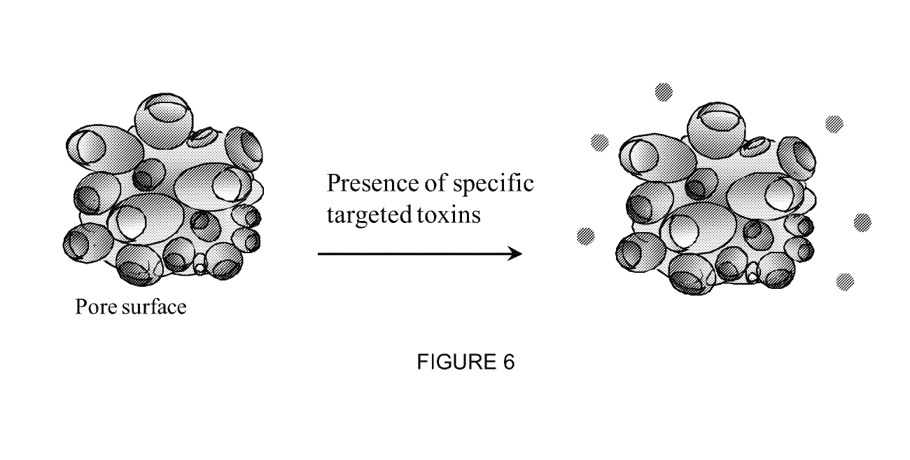

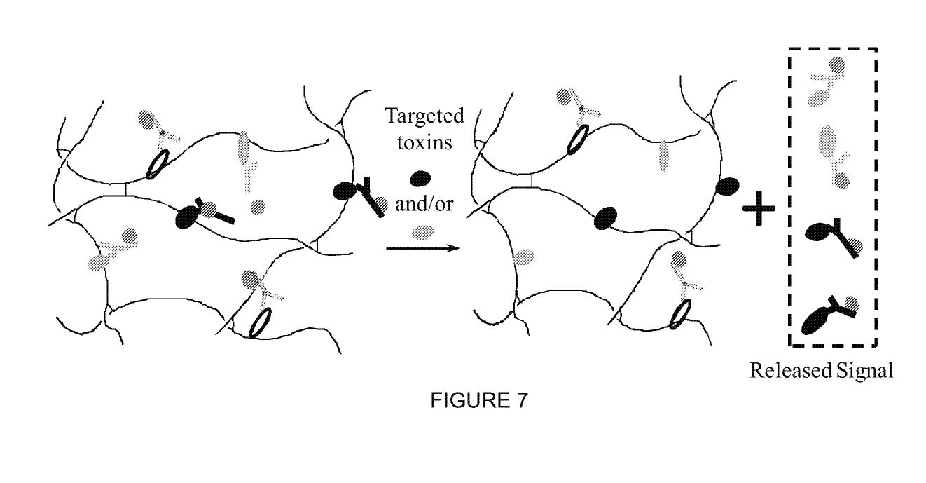

Referring to FIGS. 6 and 7, different fluorescently tagged antibodies may be provided as part of the sensor to allow for the detection of more than one target at a time.

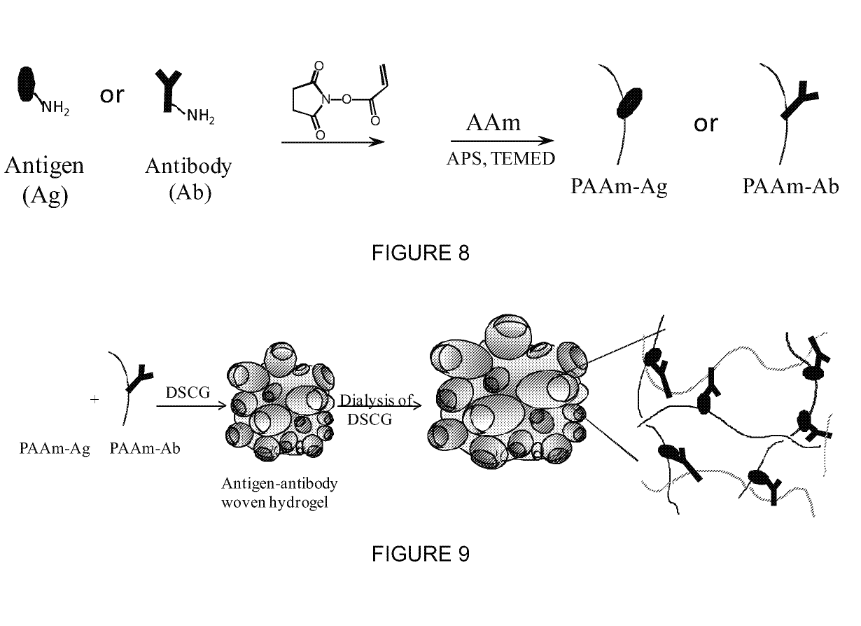

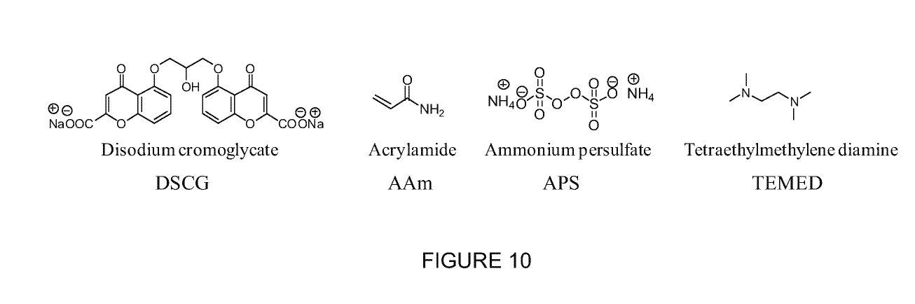

In a second embodiment of the present invention, both the antigens and antibodies used in the sensor of the present invention are covalently bonded to the gel material, as seen in FIGS. 8-10, with the non-covalent binding between the antigen and antibody functioning as the sole cross-linker for holding the gel in shape. As a result, non-covalent cross linker will be displaced when the targeted analyte is in the solution, thereby causing the gel to dissolve. This dissolution is readily visible by the eyeball, and provides a label-free, instrument-free and real-time direct “yes” and “no” detection for the targeted toxin.

Proteins (antibody and antigen) were first modified with a polymerizable acrylamide group as a monomer for making linear polyacrylamides with either antibody or antigen side chains. This modification of proteins was done by coupling the lysine groups of the proteins with N-succimidylacrylate (NSA) in phosphate buffer saline (PBS, 10 mM, pH 7.4) at 25° C. for 1 h. The acryloyl-modified antigen/antibody was copolymerized with Acrylamide (AAm) monomer to generate a linear polyacrylamide with a small percentage of protein side chains (PAAm-ag/PAAm-Ab) by mixing initiator APS and catalyst TEMED with the two monomers at 25° C. for 3 h. Polymers PAAm-Ag and PAAm-Ab were then mixed with disodium cromoglycate (DSCG). Strong affinity and binding of antigen and antibody forms noncovalent crosslinks, which results in woven hydrogels that contains pores encapsulated with water-solvated DSCG. The DSCG was removed through diffusion by soaking the woven hydrogel in PBS buffer. This dialysis generated a swollen porous hydrogel with noncovalent crosslinkers of antigen-antibody binding.

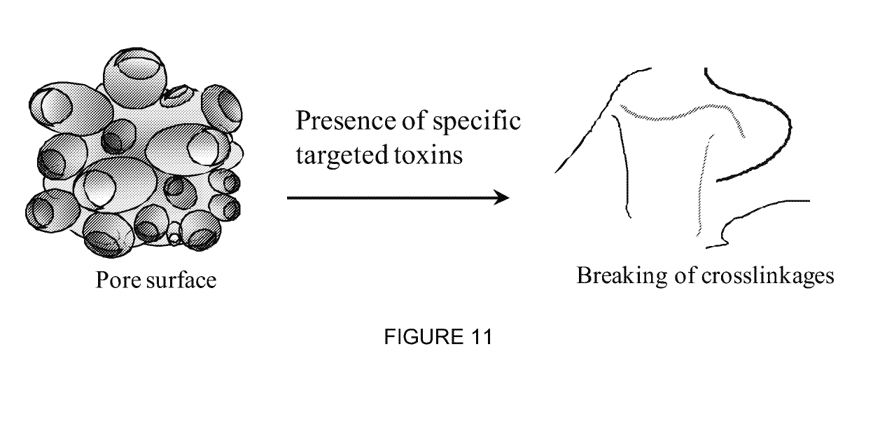

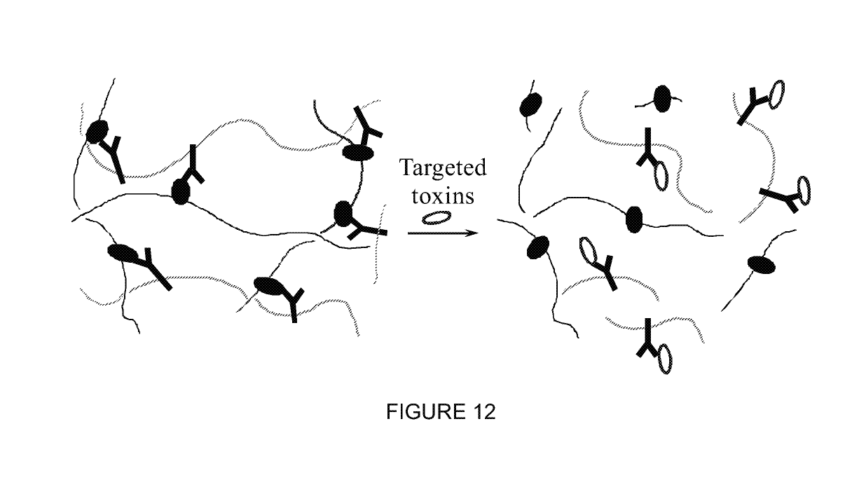

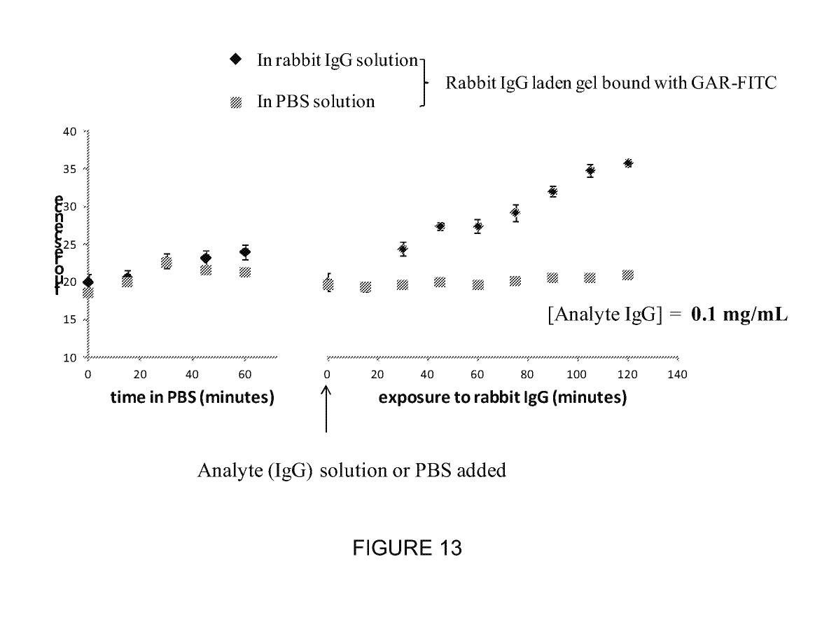

Referring to FIGS. 11 and 12, the resulting antigen-antibody woven porous hydrogel will dissolve in the presence of a targeted toxin due to displacement of the non-covalent cross-linkers in the porous gel. As seen in FIG. 13, the dissolving of the gel is highly selective and, as explained above, is readily apparent to the naked eye.

The biosensor according to the present invention can be used for the detection of a wide variety of infectious diseases including, but not limited to, HIV, Aids, tuberculosis, poliomyelitis, syphilis, Chlamydia, gonorrhea, pertussis, diphtheria, measles, tetanus, meningitis, hepatitis A, hepatitis B, hepatitis C, malaria, trypanosomiasis, chagas disease, schistosomiasis, leishmaniasis, lymphatic filariasis, onchocerciasis, leprosy, dengue, Japanese encephalitis, trachoma, ascariasis, trichuriasis, hookworm disease otitis media, respiratory infections, H5N1, H1N1, anthrax, avian influenza, swine influenza, Crimean-Congo haemorrhagic fever, Ebola, Hendra Virus, Influenza, Lassa fever, Marburg haemorrhagic fever, meningococcal disease, human monkeypox, Nipah Virus, plague, rift valley fever, smallpox, tularaemia, yellow fever, MRSA, Acinetobacter infections, Acinetobacter baumannii, Actinomycosis, Actinomyces israelii, Actinomyces gerencseriae, Propionibacterium propionicus, Amebiasis, Entamoeba histolytica, Amoebic dysentery, Anaplasmosis, Anaplasma genus, Anthrax, Bacillus anthracis, Arcanobacterium haemolyticum infection, Arcanobacterium haemolyticum, Ascariasis, Ascaris lumbricoides, Aspergillosis, Aspergillus genus, Astrovirus infection, Astroviridae family Babesiosis, Bacterial vaginosis (BV), Bacteroides infection, Clostridium botulinum, Brazilian hemorrhagic fever, Buruli ulcer Mycobacterium, ulcerans Calicivirus infection (Norovirus and Sapovirus), Caliciviridae family, Candidiasis (Moniliasis; Thrush), Chlamydophila pneumoniae infection, Chlamydophila pneumonia, Clostridium difficile infection, Bunyaviridae family, Hepatitis A Virus, Hepatitis B Hepatitis B Virus, Hepatitis C Virus, Hepatitis D Virus, Hepatitis E Virus, and Herpes simplex virus 1 and 2 (HSV-1 and HSV-2).

The biosensors according to the present invention may be used for detecting bioterrorism agents, including but not limited to Tularemia, Anthrax, Bacillus anthracis, Smallpox, Botulism, Botulinum Toxin, Clostridium botulinum, bubonic pague, Yersinia pestis, Viral hemorrhagic fevers, Arenaviruses, Lassa virus, lassa fever, junin virus, Argentine hemorrhagic fever, Machupo virus, Bolivian hemorrhagic fever, Guanarito virus, Venezuelan hemorrhagic fever, Sabia, Brazilian hemorrhagic fever, Ebola virus, Marburg virus, Brucella, brucellosis, burkholderia mallei, burkholderia pseudomallei, chalmydia psittaci, Cholera, Vibrio cholera, Clostridium perfringens, Epsilon toxin, Coxiella burnetii, Q fever, E. coli O157:H7, Nipah virus, hantavirus, Escherichia coli O157:H7, Salmonella species, Salmonella Tpyhi, typhoid fever, salmonellosis, Shigella, Shigellosis, Francisella tularensis, tularemia, Glanders, Melioidosis, Yersinia pestis, Psittacosis, Chlamydia psittaci, Ricin toxin, Ricinus communis, castor beans, Rickettsia prowazekii, typhus fever, variola major, staphylococcal enterotoxin B, viral encephalitis, alphaviruses, Venezuelan equine encephalitis, eastern equine encephalitis, Vibrio cholera, and Cryptosporidium parvun.

The biosensors according to the present invention can be used for the detection of water-borne toxins, including but not limited to Lenionella, legionellosis, Giardia Lamblia, coliform bacteria, Cryptosporidium, E. Coli, microcystin, Typhoid fever, Salmonella typhi, Cholera, Vibrio cholera, cyanobacterial toxins, Anabaena, Oscillatoria, Nodularia, Nostoc, Cylindrospermopis, Umezaka, Aphanizomenon, Cylindroapermopsis raciborski, blue-green algae, Anaemia, Arsenicosis, Ascariasis, Campylobacteriosis, Dengue, Fluorosis, Hepatitis, Japanese Encephaltis, Leptospirosis, Malria, Methaemoglobinemia, Onchocerciasis, Ringworm, Tinea, Scabie, Schistomsomiasis, Trachoma, and Paratyphoid enteric fevers.

What is claimed is:

1. A sensor for detecting a target compound, comprising:
a porous hydrogel having a first water-soluble monomer copolymerized with a second water-soluble polymer, wherein some of said second polymer is covalently bound to an antigen indicative of said target compound and some of said second monomer is covalently bound to an antibody to said antigen, and wherein said hydrogel is non-covalently cross-linked by said antigen and said antibody.

2. The sensor of claim 1, wherein the hydrogel comprises a linear polyacrylamide.

3. The sensor of claim 1, wherein said first monomer comprises acrylamide.

4. The sensor of claim 1, wherein said second monomer comprises N-succinimidyl acrylate.

5. The sensor of claim 1, wherein the presence of an antigen results in dissolution of the hydrogel.

6. A method of detecting a target compound, comprising the steps of:
providing a hydrogel having a first water-soluble monomer copolymerized with a second water-soluble monomer this is coupled to an antigen indicative of said target compound;
introducing a sample to be tested for the presence of said target compound to said hydrogel; and
observing said hydrogel to determine whether said hydrogel has undergone a change in the presence of said sample.

7. The method of claim 6, wherein said second monomer comprises N-succinimidyl acrylate and is non-covalently coupled to said antigen.

8. The method of claim 7, wherein said hydrogel further comprises a fluorescently-tagged antibody of said antigen immobilized in said hydrogel.

9. The method of claim 8, wherein said change to said hydrogel comprises the release of at least some of said fluorescently-tagged antibody in the presence of said sample.

10. The method of claim 6, wherein some of said second polymer is covalently bound to an antigen indicative of said target compound and some of some of said second monomer is covalently bound to an antibody to said antigen, and wherein said hydrogel is non-covalently cross-linked by said antigen and said antibody.

11. The method of claim 10, wherein said second monomer comprises N-succinimidyl acrylate.

12. The method of claim 11, wherein said change comprises said hydrogel at least partially dissolving in the presence of said sample.
